(19) United States
(12) Patent Application Publication
(10) Pub. No.: US 2008/0085257 A1
(43) Pub. Date: Apr. 10, 2008

(54) Composition and method for topical treatment of skin conditions

(76) Inventor: Ali Moiin, Grosse Pointe Park, MI (US)

Correspondence Address: BLISS MCGLYNN, P.C., 2075 WEST BIG BEAVER ROAD, SUITE 600, TROY, MI, 48084, US

(21) Appl. No.: 11/543,741

(22) Filed: Oct. 5, 2006

(51) Int. Cl. A61K 8/36; A61K 31/19

(52) U.S. Cl. 424/70.22; 514/557; 514/574

(57) ABSTRACT

A composition for topically treating skin conditions consists essentially of a mixture of an amount of tricarboxylic acid, an amount of glycolic acid, and alcohol as the remainder of the composition. A method for treating skin conditions includes the steps of topically applying an effective amount of the composition to an area of skin affected with a condition; leaving the composition so applied for a predetermined amount of time; rinsing the composition so applied from the affected area of skin; and so applying, leaving, and rinsing the composition at a predetermined rate to improve the condition.

### BACKGROUND OF THE INVENTION

1. Field of the Invention

The invention relates, generally, to a treatment for skin conditions and, more particularly, to a composition and method using the composition for topical treatment of such conditions.

2. Description of the Related Art

Conditions and diseases of human skin or tissue—such as acne, dryness and itchiness, stretch marks, sun damage, and wrinkles—are typically treated by topical application of any of a variety of known compositions. In turn, each of these compositions consists of active ingredients.

For example, hydroxy acids are common active ingredients in creams and lotions used to reduce wrinkles in skin by penetrating the skin and/or removing dry outer layers of the skin. More specifically, hydroxy acids, such as glycolic acid (an alpha-hydroxy acid) and lactic acid, are commonly used as ingredients in cosmetics used to reduce wrinkles spots, and other signs of aging. The hydroxy acids provide cosmetic benefits by penetrating dry, keratinized outer layers of skin, which can be defoliated in the process to reveal softer, smoother, moister, and more resilient skin located beneath the outer layers. Such penetration can open spaces between corresponding adjacent skin cells to allow more and better access to inner layers of the skin.

However, the known compositions used to topically treat skin conditions generally consist of, along with hydroxy acid(s), many other active ingredients and stabilizing agents. In particular, most of these compositions consist of at least five ingredients, and many consist of at least ten ingredients. As a result, these compositions are relatively structurally complex. In turn, they are relatively complicated, expensive, labor-intensive, and time-consuming and, thereby, inefficient to manufacture. Consequently, these compositions cost relatively much for their respective ultimate users to purchase, thus, and use. Furthermore, use of some of these compositions, even consisting of the relatively great number of respective ingredients that they do, is relatively ineffective in treating the skin conditions that they are intended to treat.

Thus, there is a need for a composition for topically treating skin conditions that consists of fewer ingredients relative the respective known compositions used to topically treat skin conditions. There is a need for such a composition that also is more structurally simple relative such known compositions. There is a need for such a composition that is also less complicated, expensive, labor-intensive, and time-consuming and, thereby, less inefficient to manufacture relative such known compositions. There is a need for such a composition that also costs less for its ultimate users to purchase and, thus, use relative such known compositions. There is a need for such a composition also use of which is more effective in treating the skin conditions that it is intended to treat relative such known compositions. There is a need for also a method for using such composition.

### SUMMARY OF THE INVENTION

The invention overcomes the disadvantages in the related art in a composition and method for topically treating skin conditions. The composition consists essentially of a mixture of an amount of tricarboxylic acid, an amount of glycolic acid, and a stabilizing agent as the remainder of the composition. The method includes the steps of topically applying an effective amount of the composition to an area of skin affected with a condition; leaving the composition so applied for a predetermined amount of time; rinsing the composition so applied from the affected area of skin; and so applying, leaving, and rinsing the composition at a predetermined rate to improve the condition.

One advantage of the composition for topically treating skin conditions of the present invention is that the composition consists of fewer ingredients relative the respective known compositions used to topically treat skin conditions.

Another advantage of the composition for topically treating skin conditions of the present invention is that the composition is more structurally simple relative such known compositions.

Another advantage of the composition for topically treating skin conditions of the present invention is that the composition is less complicated, expensive, labor-intensive, and time-consuming and, thereby, less inefficient to manufacture relative such known compositions

Another advantage of the composition for topically treating skin conditions of the present invention is that the composition costs less for its ultimate users to purchase and, thus, use relative such known compositions.

Another advantage of the composition for topically treating skin conditions of the present invention is that use of the composition is more effective in treating the skin conditions that it is intended to treat relative use of such known compositions.

Another advantage of the composition for topically treating skin conditions of the present invention is that use of the composition can provide cosmetic benefits to the skin.

Another advantage of the composition for topically treating skin conditions of the present invention is that use of the composition can be effective on substantially all skin types.

Other objects, features, and advantages of the composition for topically treating skin conditions of the present invention will be readily appreciated as the composition and method become more understood while reading the subsequent description of the composition and method.

### DETAILED DESCRIPTION OF AN EMBODIMENT OF THE INVENTION

A composition and method for topically treating skin conditions of the invention is described below. The composition and method are intended to be used in connection with treating skin conditions and/or diseases such as, but not limited to, acne, dryness and itchiness, stretch marks, sun damage, and wrinkles. As such, it should be appreciated by those having ordinary skill in the related art that the composition and method can be used in connection with treating any suitable skin condition and/or disease. It should be so appreciated also that, although the composition and method find special application in connection with their topical treatment of human facial conditions, the composition and method can find application in connection with their topical treatment of conditions of any suitable skin of the human body.

Treating a condition topically with the composition provides clear benefits, such as ease of delivery of the composition, lower purification requirements for each of the active ingredients of the composition, less stringent regulatory requirements, and ability to target the composition directly to specific sites affected with a condition.

The composition consists essentially of a mixture (or blend) of an amount of tricarboxylic acid, an amount of glycolic acid, and a stabilizing agent as the remainder of the composition. The glycolic acid complements activities of the tricarboxylic acid by increasing absorption by the treated skin and enhancing penetration into tissues of the treated skin of the composition and, thereby, optimizing effectiveness of the composition. The glycolic acid also can provide cosmetic benefits to the skin.

In a preferred embodiment of the composition, the amount of tricarboxylic acid is between about 5% and about 14% by volume of the composition, and the amount of glycolic acid is between about 15% and about 35% by volume of the composition. The stabilizing agent is alcohol. These three ingredients are blended into a conventional solution as a carrier for its topical application to the skin to treat the conditions of the skin. In a more preferred embodiment of the composition, the amount of tricarboxylic acid is between about 10% and about 14% by volume of the composition, and the amount of glycolic acid is between about 25% and about 35% by volume of the composition. The alcohol is benzyl alcohol. In a most preferred embodiment of the composition, the amount of tricarboxylic acid is about 11% by volume of the composition, and the amount of glycolic acid is about 30% by volume of the composition.

Each of the tricarboxylic and glycolic acids is an alpha hydroxy acid and, thereby, as mentioned above, complements respective activities of the other ingredients of the composition, e.g., increases absorption by the treated skin and enhances penetration into tissues of the treated skin of the composition. In this way, effectiveness of the composition is optimized. In addition, as alpha hydroxy acids, the tricarboxylic and glycolic acids can provide cosmetic benefits to the treated skin.

It should be appreciated by those having ordinary skill in the related art that any suitable stabilizing agent or combination of stabilizing agents can be used for the composition. By way of example only, the stabilizing agent or combination of stabilizing agents can include any suitable organic or inorganic substance or combination of organic and/or inorganic substances—such as, but not limited to, salts, water, saline, buffers, vegetable and mineral oils, cyclodextrin, hydroxypropylcyclodextrin (especially beta-type), polyethylene glycols, glycerol triacetate and other fatty-acid glycerides, gelatin, soya lecithin, carbohydrates (such as lactose, starch, or other sugars), magnesium stearate, talc, cellulose, and/or the like. It should be so appreciated also that the three ingredients can be blended into a conventional cream, lotion, astringent, or gel as a carrier for its topical application to the skin to treat the conditions of the skin.

The method for treating skin conditions includes the steps of topically applying an effective amount of the composition to an area of skin affected with a condition; leaving the effective amount so applied for a predetermined amount of time; rinsing the composition so applied from the affected area of skin; and so applying, leaving, and rinsing the composition at a predetermined rate to improve the condition. The effective amount is the quantity of the composition that produces the desired effect, e.g., ameliorates or improves the condition.

In a preferred embodiment of the method, the effective amount of the composition is left topically applied to the area of skin affected with the condition for no fewer than about two minutes and no greater than about twelve minutes. Also, the composition so applied is denatured, or rinsed from the affected area of skin, with water. And, the composition is so applied, left, and rinsed (denatured) at a rate of about once every two weeks for about twelve weeks for a total of about six applications to improve the condition.

More preferably, the composition is left so applied for about two minutes for the first application, about four minutes for the second application, about six minutes for the third application, about eight minutes for the fourth application, about ten minutes for the fifth application, and about twelve minutes for the sixth application. However, such effective amount, amount of time, and rate depend upon such factors as the particular condition being treated, degree of severity of the particular condition, and degree of sensitivity of the affected area of skin.

As such, it should be appreciated by those having ordinary skill in the related art that the effective amount of the composition to be topically applied to the area of skin affected with the condition can be any suitable amount. It should be so appreciated also that the composition can be left so applied for any suitable amount of time. It should be so appreciated also that the composition so applied can be denatured, or rinsed from the affected area of skin, with any suitable substance. It should be so appreciated also that the composition can be so applied, left, and rinsed at any suitable rate to improve the condition.

Use of the composition can be effective on substantially all skin types. However, use of acids, in general, and hydroxy acids, in particular, can have undesirable side effects—including blistering, burning, itching, rash, severe redness, skin discoloration, and swelling. Accordingly, in using embodiments of the composition consisting of a relatively high concentration of the acids, relatively more care should be taken when the effective amount of the composition is topically applied to the affected area of skin, particularly if such area is relatively sensitive. In such a case, the acidic concentration can be gradually escalated to avoid adverse reactions of the treated area.

The composition consists of fewer ingredients relative the respective known compositions used to topically treat skin conditions. Also, the composition is more structurally simple relative such known compositions. And, the composition is less complicated, expensive, labor-intensive, and time-consuming and, thereby, less inefficient to manufacture relative such known compositions. Furthermore, the composition costs less for its ultimate users to purchase and, thus, use relative such known compositions. In addition, use of the composition is more effective in treating the skin conditions that it is intended to treat relative use of such known compositions. Moreover, use of the composition can provide cosmetic benefits to the skin. Plus, use of the composition can be effective on substantially all skin types.

The composition and method have been described in an illustrative manner. It is to be understood that the terminology that has been used is intended to be in the nature of words of description rather than of limitation. Many modifications and variations of the composition and method are possible in light of the above teachings. Therefore, within the scope of the appended claims, the composition and method may be practiced other than as specifically described.

What is claimed is:

1. A composition for topically treating a skin condition consisting essentially of a mixture of:
an amount of tricarboxylic acid;
an amount of glycolic acid; and
a stabilizing agent as the remainder of the composition.

2. The composition as set forth in claim 1, wherein the amount of tricarboxylic acid is between about 5% and about 14% by volume of the composition.

3. The composition as set forth in claim 2, wherein the amount of tricarboxylic acid is about 11% by volume of the composition.

4. The composition as set forth in claim 1, wherein the amount of glycolic acid is between about 15% and about 35% by volume of the composition.

5. The composition as set forth in claim 4, wherein the amount of glycolic acid is about 30% by volume of the composition.

6. The composition as set forth in claim 1, wherein the stabilizing agent is alcohol.

7. The composition as set forth in claim 6, wherein the alcohol is benzyl alcohol.

8. The composition as set forth in claim 1, wherein the skin condition is selected from the group consisting of acne, dryness and itchiness, stretch marks, sun damage, and wrinkles.

9. A method for treating skin conditions comprising the steps of:
topically applying an effective amount of a composition to an area of skin affected with a condition;
leaving the composition so applied for a predetermined amount of time;
rinsing the composition so applied from the affected area of skin; and
so applying, leaving, and rinsing the composition at a predetermined rate to improve the condition;
wherein the composition consists essentially of a mixture of:
an amount of tricarboxylic acid;
an amount of glycolic acid; and
alcohol as the remainder of the composition.

10. The method as set forth in claim 9, wherein the amount of tricarboxylic acid is between about 5% and about 14% by volume of the composition.

11. The method as set forth in claim 10, wherein the amount of tricarboxylic acid is about 11% by volume of the composition.

12. The method as set forth in claim 9, wherein the amount of glycolic acid is between about 15% and about 35% by volume of the composition.

13. The method as set forth in claim 12, wherein the amount of glycolic acid is about 30% by volume of the composition.

14. The method as set forth in claim 9, wherein the stabilizing agent is alcohol.

15. The method as set forth in claim 14, wherein the alcohol is benzyl alcohol.

16. The method as set forth in claim 9, wherein the skin condition is selected from the group consisting of acne, dryness and itchiness, stretch marks, sun damage, and wrinkles.

17. The method as set forth in claim 9, wherein the composition so applied is rinsed from the affected area of skin with water.

18. The method as set forth in claim 9, wherein the composition is left so applied for no fewer than about two minutes and no greater than about twelve minutes.

19. The method as set forth in claim 18, wherein the composition is so applied, left, and rinsed at a rate of about once every two weeks for about twelve weeks for a total of about six applications to improve the condition.

20. The method as set forth in claim 19, wherein the composition is left so applied for about two minutes for the first application, about four minutes for the second application, about six minutes for the third application, about eight minutes for the fourth application, about ten minutes for the fifth application, and about twelve minutes for the sixth application.
